(19) United States
(12) Patent Application Publication
(10) Pub. No.: US 2005/0027208 A1
(43) Pub. Date: Feb. 3, 2005

(54) Menstrual cycle monitoring apparatus, toilet apparatus, and menstrual cycle monitoring method

(76) Inventors: Takako Shiraishi, Ikoma-shi (JP); Hirosi Yamamoto, Shijonawate-shi (JP); Manami Kaida, Nara-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/899,159

(22) Filed: Jul. 27, 2004

(30) Foreign Application Priority Data
Jul. 28, 2003 (JP) 2003-281276

(51) Int. Cl. A61B 10/00

(52) U.S. Cl. 600/551

(57) ABSTRACT

A menstrual cycle monitoring apparatus 100 that determines a menstrual cycle of a user is composed of: a clock unit 103 that measures a usage quantity (i.e., a cleansing time period taken from when a bidet switch 111 is pressed to when a stop switch 113 is pressed) of the bidet apparatus 116 that cleanses the vaginal area; a memory unit 104 that accumulates a history of the measured usage quantity for each date; and a menstrual cycle determining unit 105 that determines the menstrual cycle of the user on the basis of the history of the usage quantity accumulated in the memory unit 104.

### BACKGROUND OF THE INVENTION

(1) Field of the Invention

The present invention relates to a monitoring apparatus and a monitoring method for determining a menstrual cycle, including menstruation and ovulation of a female user, on the basis of a usage history for a bidet apparatus mounted on a toilet fixture to cleanse the rectal or genital area of the human body.

(2) Description of the Related Art

For women, menstruation is an information source to know about their physical condition. Hormones released from the brain and the pituitary gland stimulate the function of the ovaries which, in turn, secrete two female hormones, a follicular hormone (also called estrogen) and a luteinizing hormone. These secreted hormones then stimulate buildup of the endometrium, causing menstruation. In other words, the rhythm of menstruation indicates the rhythm of hormones. The hormone imbalance causes various medical problems and diseases. For this reason, the management of the menstrual cycle is essential for a female health care. Many women experience uncomfortable symptoms before and/or after menstruation. Especially for such a woman, it is very important to understand her own menstrual cycle and to know the date of the onset of her next menstruation in advance for planning her schedule of business, housework, and leisure.

In general, a woman can determine her menstrual cycle for herself by recording the dates of the onset of her menstruation in her schedule book, calendar, or electronic organizer so as also to manage and predict her menstrual cycle. Or, as a well-known conventional method, she can record her basal body temperature to know her ovulation date and menstrual cycle. Also, there is a conventional apparatus that automatically records the measurement data obtained through a basal body thermometer and calculates a menstrual or ovulation cycle of a female user on the basis of the recorded basal body temperature data. Using the firstly mentioned method, however, the woman may forget to write down the date of the onset of menstruation. As to the secondly mentioned method, the measurement of the basal body temperature itself is burdensome. Meanwhile, although the measurement data is automatically recorded using the above conventional apparatus, the burdensome measurement of the basal body temperature is still a problem. Under such a method by which the measurement of the basal body temperature is required, the menstrual cycle can be determined only after observing the fluctuations in the basal body temperature over a period of time as long as a few months. Nonetheless, the measurement itself is frequently forgotten and thus it is too difficult to keep observing.

Japanese Laid-Open Patent Application No. H9-75352 discloses a female physical condition managing apparatus that is provided in a toilet space to detect the female user's menstrual blood in urine during urination so as to predict her menstrual cycle including the dates of her next ovulation and the onset of her next menstruation. Using this apparatus, the female user can find her menstrual cycle without having to take the burdensome measurement of her basal body temperature.

Moreover, Japanese Laid-Open Patent Application No. H11-84036 discloses a technology to reduce the inconvenience of measuring the basal body temperature. According to this technology, a female user is notified of her ovulation date that is calculated on the basis of the record obtained through a temperature sensor embedded in a wristwatch.

Using the former invention, however, a toilet bowl or seat has to be additionally equipped with: an apparatus for collecting urine and rinse water; a test reagent used for detecting blood reaction of the collected bodily fluid; and a special apparatus such as an odor sensor. In this way, this invention is costly and also narrows a space around the toilet bowl due to the upsized equipment.

Meanwhile, using the latter invention, the female user has to wear the special watch each day. In addition to a possibility that she may forget to wear the watch, women's psychology that they want to change their watches depending on time, place, and occasion cannot be responded.

### SUMMARY OF THE INVENTION

The present invention has been conceived in view of the above problem, and it is an object of this invention to provide a menstrual cycle monitoring apparatus and a menstrual cycle monitoring method which are capable of easily determining a menstrual cycle of a female user without having to measure the basal body temperature and upsize the toilet fixture.

The object can be achieved by a menstrual cycle monitoring apparatus of the present invention that determines a menstrual cycle of a user, the apparatus is made up of: a usage quantity measuring unit operable to measure a usage quantity of a bidet apparatus that cleanses a vaginal area of the user; a history accumulating unit operable to accumulate a history of the measured usage quantity for each date; and a menstrual cycle determining unit operable to determine the menstrual cycle of the user on the basis of the history of the measured usage quantity accumulated in said history accumulating unit.

Using this apparatus of the present invention, the female user can easily know about her menstrual cycle without having to upsize the toilet fixture or take the burdensome measurement of the basal body temperature, and can also take control of her health care and schedule management on the basis of the determined menstrual cycle.

Moreover, the object can be achieved by the menstrual cycle monitoring apparatus of the present invention, wherein the usage quantity is an amount of water used by said bidet apparatus for a cleansing, said usage quantity measuring unit includes a water meter operable to measure the amount of water used for the cleansing, said history accumulating unit accumulates a history of the measured amount of water, said menstrual cycle determining unit includes a menstrual day determining unit operable to determine a possible menstrual day by reference to the history of the measured amount of water, and said menstrual cycle determining unit determines the menstrual cycle of the user on the basis of a determination result given by said menstrual day determining unit.

Also, said menstrual cycle determining unit may further include an onset date determining unit operable to determine a date of an onset of a menstruation of the user by reference to the history in accordance with a number of consecutive days that have been determined as possible menstrual days by said menstrual day determining unit, and said menstrual cycle determining unit may determine the menstrual cycle of the user on the basis of a determination result given by said onset date determining unit.

Also, said menstrual cycle determining unit may determine the menstrual cycle of the user in accordance with a length of an interval between a date of an onset of a menstruation and a date of an onset of a next menstruation, with both of these dates having been accumulated in the history and having been determined by said onset date determining unit.

Moreover, the menstrual cycle monitoring apparatus may further include an onset date predicting unit operable to predict a date of an onset of a future menstruation of the user on the basis of a determination result given by said menstrual cycle determining unit.

Furthermore, the menstrual cycle monitoring apparatus may further include an ovulation date predicting unit operable to predict a future ovulation date of the user on the basis of a prediction result given by said onset date predicting unit.

It should be noted that the present invention can be realized not only as the described menstrual cycle monitoring apparatus, but also as a menstrual cycle monitoring method that performs steps corresponding to the stated characteristic units provided for the menstrual cycle monitoring apparatus or as a program that causes a computer to execute these steps. Also, it should be understood that such program can be distributed via recording medium such as a CD-ROM as well as via transmission medium such as the Internet.

According to the menstrual cycle monitoring apparatus and method of the present invention, no special measurements are required to determine the menstrual cycle in the bathroom. In other words, the menstrual cycle can be determined without upsizing the toilet fixture. Also, the female user is not bothered to measure her morning basal body temperature. All she has to do is inputting her own identifying information when using the bidet function. By doing so, the female user can know her menstrual cycle, the date of the onset of her next menstruation, and the date of her next ovulation then and there. Thus, the female user can obtain information useful for taking control of her health care, birth control, and schedule management.

### BRIEF DESCRIPTION OF THE DRAWINGS

These and other objects, advantages and features of the invention will become apparent from the following description thereof taken in conjunction with the accompanying drawings that illustrate a specific embodiment of the invention. In the Drawings:

FIG. 1 is a function block diagram showing each construction of a bidet apparatus and a menstrual cycle monitoring apparatus of an embodiment of the present invention;

FIG. 2 is an external view of a remote controller and a main unit of the bidet apparatus on which the menstrual cycle monitoring apparatus shown in FIG. 1 is mounted;

FIG. 3 is an external view of the remote controller shown in FIG. 2 and also shows an example of a user selection screen displayed on a liquid crystal display of the remote controller;

FIG. 4 shows an example of a nozzle position setting screen displayed on the liquid crystal display of the remote controller shown in FIG. 3;

FIG. 5 is a flowchart showing an operation performed by the menstrual cycle monitoring apparatus shown in FIG. 1, from determining a menstrual cycle of the user to indicating the determination result to the user;

FIG. 6 is a graph representing a calculation method used by a menstrual cycle determining unit for calculating a cleansing time period t0 spent by the user on cleansing using the bidet function;

FIG. 7 shows an example of usage data stored in a memory unit shown in FIG. 1;

FIG. 8 is a graph showing respective typical bidet usage patterns during menstruation and other days;

FIG. 9A is an example of a table showing determination standards that the menstrual cycle determining unit refers to when determining the menstrual possibility for each day on the basis of an average cleansing time period t3;

FIG. 9B is an example of a table showing determination standards that the menstrual cycle determining unit refers to when determining the date of the onset of the user's menstruation on the basis of a menstrual calendar shown in FIG. 10;

FIG. 10 is an example of a menstrual calendar created in the memory unit shown in FIG. 1;

FIG. 11 is a flowchart showing an operation performed by the menstrual cycle determining unit to determine the date of the onset of the user's menstruation by reference to the table shown in FIG. 9B;

FIG. 12 is an example of a table showing determination standards that a menstrual cycle predicting unit refers to when predicting the date of the onset of the user's next menstruation on the basis of descriptions in the column of the onset date of menstruation of the menstrual calendar covering the last 50 days;

FIG. 13 is a flowchart showing an operation performed by the menstrual cycle predicting unit to predict the date of the onset of the user's next menstruation according to the table shown in FIG. 12; and

FIG. 14 is an example of a calendar showing prediction results given by an ovulation date predicting unit which is additionally provided for the menstrual cycle monitoring apparatus shown in FIG. 1.

### DESCRIPTION OF THE PREFERRED EMBODIMENT

The following is a description of an embodiment according to the present invention, with reference to the drawings.

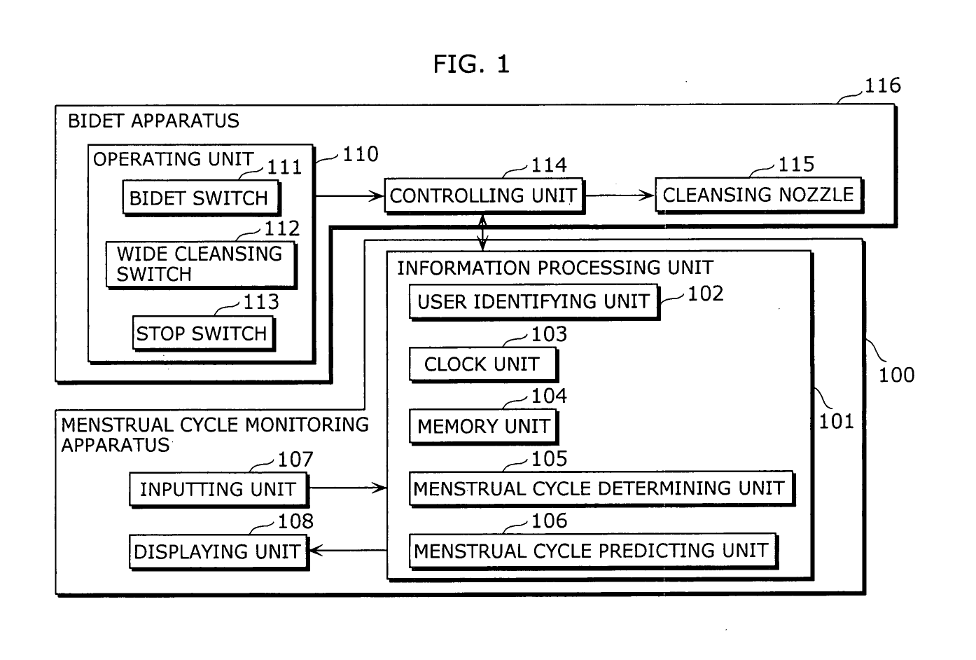

FIG. 1 is a function block diagram showing each construction of a bidet apparatus 116 and a menstrual cycle monitoring apparatus 100 of the embodiment of the present invention. The bidet apparatus 116 shown in FIG. 1 is set to a toilet bowl, for example, and sprays a stream of warm water from a cleansing nozzle mounted on the toilet bowl or seat after urination or defecation so as to cleanse the vaginal area of a female user. The bidet apparatus 116 is used by the female user to rinse the vaginal area after urination and to cleanse menstrual blood or vaginal discharge from the area. This bidet apparatus 116 is composed of an operating unit 110, a controlling unit 114, and a cleansing nozzle 115. The operating unit 110 is an inputting unit used by the user when directing the bidet apparatus 116 to start a cleansing or specifying a cleansing way, and it is provided with operation switches, such as a bidet switch 111, a wide cleansing switch 112, and a stop switch 113. When pressed, the bidet switch 111 outputs a signal indicating to start a cleansing by the bidet apparatus 116. The wide cleansing switch 112, when pressed, outputs a signal indicating to cleanse a wider area (referred to as the “wide cleansing” hereinafter) than the area cleansed by the general bidet function activated by the bidet switch 111. The stop switch 113, when pressed, outputs a signal indicating to stop the cleansing. The cleansing nozzle 115 is movable and usually mounted on the toilet bowl or seat, and sprays a stream of warm water, for example, from its tip. The controlling unit 114 controls an operation of the cleansing nozzle 115 as well as a supply of warm water and the like to the cleansing nozzle 115, according to a signal outputted corresponding to the operation switch that was pressed on the operating unit 110.

The menstrual cycle monitoring apparatus 100 determines a menstrual cycle of the female user according to her usage history for the bidet apparatus 116, on the basis that a period of time spent on cleansing using the bidet apparatus 116 is generally longer during menstruation than other regular days. The monitoring apparatus 100 is composed of an information processing unit 101, an inputting unit 107, and a displaying unit 108. The information processing unit 101 is provided for a main unit 220 (not shown in FIG. 1) of the bidet apparatus 116, for example, and it detects how much time the user spends on cleansing according to which selected operation switch of the bidet apparatus 116. On the basis of the cleansing way selected by the user and the detected period of time taken for the cleansing, the information processing unit 101 determines the menstrual cycle of the user. The unit 101 is provided with a user identifying unit 102, a clock unit 103, a memory unit 104, a menstrual cycle determining unit 105, and a menstrual cycle predicting unit 106. The user identifying unit 102 identifies the user of the bidet apparatus 116 according to identifying information inputted from the inputting unit 107. The clock unit 103 is provided with a calendar function for timekeeping the current date and time. The memory unit 104 previously stores tables describing methods and standard values referred to by the menstrual cycle determining unit 105 and the menstrual cycle predicting unit 106 for determining the menstrual period, the date of the onset of menstruation, and the menstrual cycle of the user. The memory unit 104 also has a storage area for accumulating a usage history, such as whether or not the bidet function has been used or a period of time taken for a cleansing, every time the user uses the bidet apparatus 116. This storage area is also used for storing determination results given by the menstrual cycle determining unit 105 and prediction results given by the menstrual cycle predicting unit 106. The menstrual cycle determining unit 105 determines a possible menstrual period on the basis of usage pattern of the user for the bidet apparatus 116, and determines the date of the onset of the user's menstruation on the basis of the consecutiveness of the determined possible menstrual days. A more detailed explanation is given as an example as follows.

First, the menstrual cycle determining unit 105 calculates a period of time taken from when the user presses the bidet switch 111 of the operating unit 110 to when the user presses the stop switch 113 (this time period is, in other words, the cleansing period of time) from the current time obtained through the clock unit 103. The menstrual cycle determining unit 105 then attaches information, such as the current date, the usage time of day, and a description as to whether the wide cleansing switch 112 has been pressed, to the calculated time period, and accumulates this whole information into the memory unit 104 for each user. Next, the menstrual cycle determining unit 105 determines the menstrual period and date of the onset of menstruation of the user according to the user's usage history accumulated in the memory unit, by reference to the tables showing various determination standards in the memory unit 104. Meanwhile, the menstrual cycle predicting unit 106 predicts the user's menstrual cycle and the date of the onset of the user's next menstruation on the basis of the menstrual period and the date of the onset of menstruation determined by the menstrual cycle determining unit 105.

The inputting unit 107 receives an input of identifying information from the user, such as a user's number, that is used for uniquely identifying the user. Also, the inputting unit 107 receives inputs of personal information of the user, including the user's age, weight, and menstrual dates. The displaying unit 108 shows information regarding the user's menstrual cycle determined or predicted by the information processing unit 101 to the user.

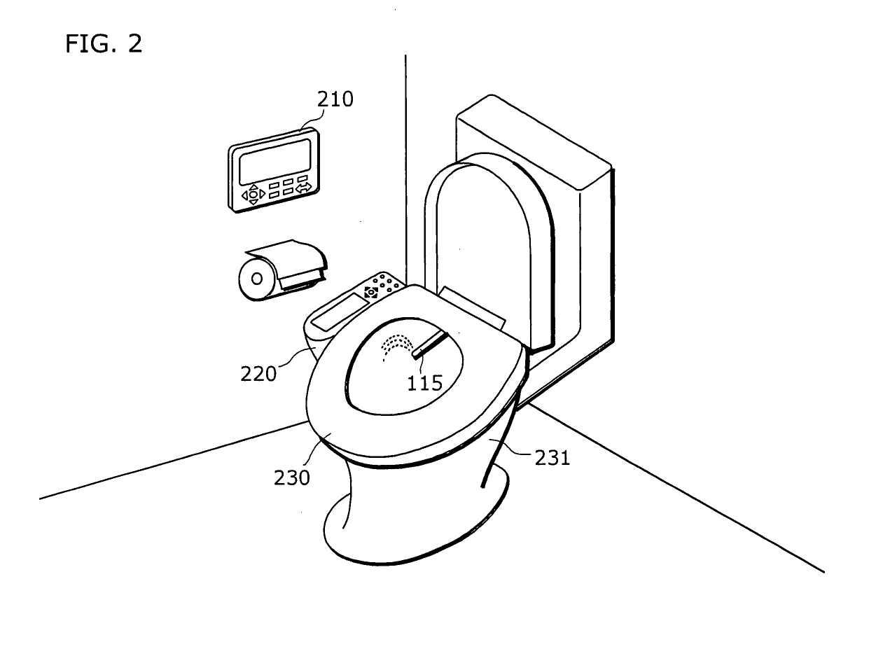

Next, an explanation is given for an operation performed by the menstrual cycle monitoring apparatus 100 when the user uses the bidet apparatus 116, with reference to FIGS. 2 to 6.

FIG. 2 is an external view of a remote controller 210 and the main unit 220 of the bidet apparatus 116 on which the menstrual cycle monitoring apparatus 100 shown in FIG. 1 is mounted. As shown in FIG. 2, a toilet seat 230 having the main unit 220 and the cleansing nozzle 115 of the bidet apparatus 116 is set to a toilet bowl 231. In a case where the menstrual cycle monitoring unit 100 is built in the main unit 220 of the bidet apparatus 116, both the controlling unit 114 of the bidet apparatus 116 and the information processing unit 101 of the menstrual cycle monitoring apparatus 100 are realized by programs stored in the memory unit 104 to be executed by a CPU of the bidet apparatus 116. Also, both the inputting unit 107 and the displaying unit 108 of the menstrual cycle monitoring apparatus 100 as well as the operating unit 110 of the bidet apparatus 116 are provided for the remote controller 210 and the main unit 220.

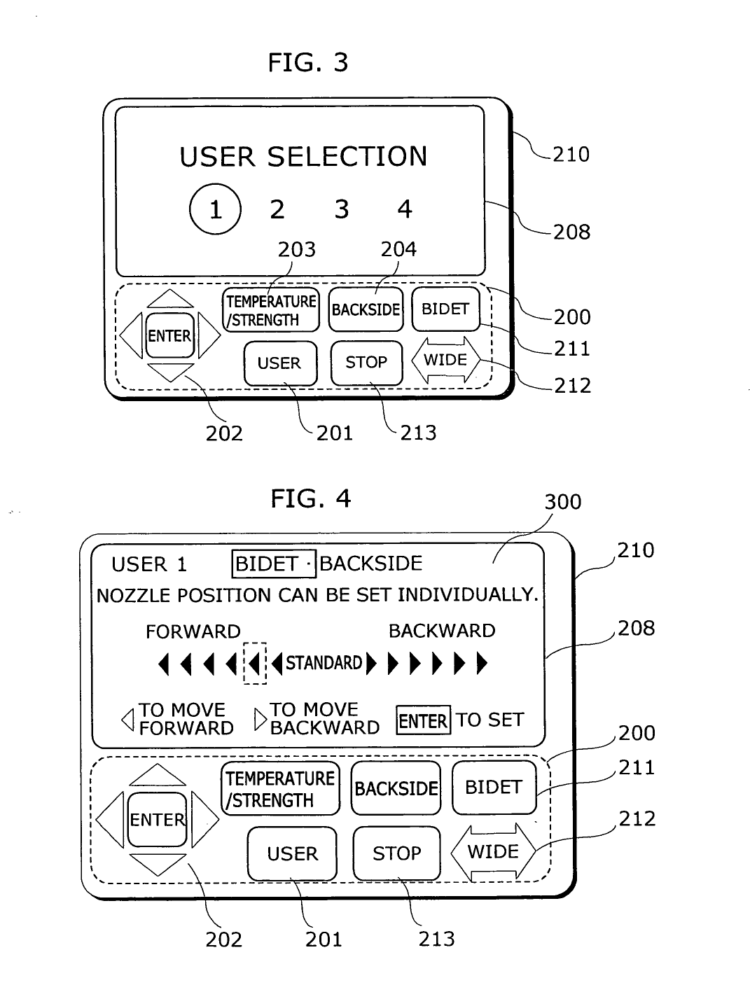

FIG. 3 is an external view of the remote controller 210 shown in FIG. 2 and also shows an example of a user selection screen displayed by the displaying unit 108 of the remote controller 210. The remote controller 210 has a function of sending a signal corresponding to an operation key that was pressed by the user to the main unit 220 via infrared or short-range radio communication. Also, the remote controller 210 is provided with an operation panel 200 and a liquid crystal display 208. The operation panel 200 has a user selection key 201, a selection enter key 202, a temperature/strength adjusting key 203, a backside cleansing key 204, a bidet key 211, a wide cleansing key 212, and a stop key 213. Both the user selection key 201 and the selection enter key 202 serve as the inputting unit 107 of the menstrual cycle monitoring apparatus 100 shown in FIG. 1. The selection enter key 202 includes four arrow keys and an enter key. The arrow keys are used for moving a cursor (indicated as “∘” in FIG. 3), and the enter key is used for selecting data corresponding to the position of the cursor.

When the user presses the user selection key 201, a signal directing to display the user selection screen is sent to the information processing unit 101. Upon receiving this signal, the information processing unit 101 reads out display data related to the user selection screen as shown in FIG. 3 from the memory unit 104, and then directs the liquid crystal display 208 (i.e., the displaying unit 108 shown in FIG. 1) to display the user selection screen. When the user presses one of the four arrow keys of the selection enter key 202 while looking at the user selection screen displayed on the liquid crystal display 208, a signal corresponding to the direction of the pressed arrow key is inputted to the information processing unit 101. Every time one of the arrow keys corresponding to the currently possible directions is pressed, the information processing unit 101 moves the cursor indicated as “∘” on the user selection screen to the direction of the pressed arrow key. For example, the female user can input her identifying information by pressing the selection enter key 202 when the cursor is positioned on a number that is previously set as her identifying number. When the selection enter key 202 is pressed, the user identifying unit 102 receives this input of the number encircled with “∘” (i.e., the cursor) as the identifying information of the female user.

Upon the input of the user's identifying information in this way, the user identifying unit 102 reads out a pre-stored individual setting of the cleansing nozzle 115, for example, from the memory unit 104 and then outputs the read setting to the controlling unit 114. In a case where the bidet key 211 is pressed, the controlling unit 114 directs the cleansing nozzle 115 to move to a cleansing position that is previously set specifically for the current user in accordance with the individual setting inputted from the user identifying unit 102. As another example, in a case where the backside cleansing key 204 is pressed, the controlling unit 114 directs the cleansing nozzle 115 to move to a backside cleansing position that is previously set specifically for the current user in accordance with the individual setting inputted from the user identifying unit 102.

FIG. 4 shows an example of a nozzle position setting screen 300 displayed on the liquid crystal display 208 of the remote controller 210 shown in FIG. 3. The nozzle position setting screen 300 shown in FIG. 4 is displayed, for example, after the user selection key 201 is pressed for two seconds or longer following the input of the identifying information from the user selection screen shown in FIG. 3. As shown in FIG. 4, “USER 1” inputted from the user selection screen is displayed at the upper left corner of the nozzle position setting screen 300. On the right side of this, both “BIDET” and “BACKSIDE” are indicated, and this example case shows that “BIDET” is currently selected. In the middle part of the liquid crystal display 208, six arrows representing six steps are displayed respectively in both the forward and backward directions with “STANDARD” being the center. These 12 arrows allow the user to adjust the cleansing position of the cleansing nozzle 115 in the six steps in each of the forward and backward directions. In this example shown in FIG. 4, a position that is two steps forward from the standard is currently selected. With this being the situation, when the selection enter key 202 is pressed, this position two steps forward from the standard is stored as the cleansing position of the cleansing nozzle 115 for the “USER 1” in the case of using the bidet function. Accordingly, the user can previously store her most preferable cleansing position of the cleansing nozzle 115, thereby obtaining an optimum cleansing effect. In addition, from the next time, the user can have the cleansing nozzle 115 move to that position simply by inputting her identifying information from the user selection screen.

The temperature/strength adjusting key 203 is not related to any operation switches included in the operating unit 110 shown in FIG. 1. When pressed once, for example, the temperature/strength adjusting key 203 outputs a signal directing to display a temperature adjustment screen. When pressed twice, on the other hand, the temperature/strength adjusting key 203 outputs a signal directing to display a warm water strength adjustment screen. As is the case with the stated processing performed by the information processing unit 101 for receiving the input of the identifying information, the controlling unit 114 directs the liquid crystal display 208 to display the temperature adjustment screen or the warm water strength adjustment screen, and receives a corresponding input, i.e., temperature information or strength information, that is selected and entered by the user through the selection enter key 202. According to the inputted temperature information, the controlling unit 114 controls a warm-water heater of a warm-water tank (both not shown) so as to maintain the warm water at the temperature indicated in the information. According to the inputted strength information, the controlling unit 114 controls a water-amount adjusting valve (not shown) so as to adjust the strength of warm water to be sprayed from the cleansing nozzle 115.

Like the temperature/strength adjusting key 203, the backside cleansing key 204 is not related to any operation switches included in the operating unit 110 shown in FIG. 1. When pressed by the user, the backside cleansing key 204 outputs a signal directing to start a backside cleansing. Following this signal, the controlling unit 114 directs the cleansing nozzle 115 to move to a position corresponding to the anal area of the user and spay warm water to cleanse the area.

The liquid crystal display 208 serves as the displaying unit 108 shown in FIG. 1, and displays the user selection screen to guide the user to input her identifying information. The liquid crystal display 208 displays a menstrual cycle of the user as well.

The bidet key 211 serves as the bidet switch 111 shown in FIG. 1, the wide cleansing key 212 as the wide cleansing switch 112, and the stop key 213 as the stop switch 113. The bidet key 211 is usually used by female users after urination and defecation. When the bidet key 211 is pressed, warm water or cleansing liquid is sprayed from the cleansing nozzle 115 mounted on the toilet bowl to the vaginal and urethral areas. To be more specific, when the bidet key 211 is pressed by the user and the identifying information has already been inputted, the controlling unit 114 directs the cleansing nozzle 115 to move to the cleansing position that is previously set by the user and to start a cleansing. When the identifying information has not been inputted, the controlling unit 114 directs the cleansing nozzle 115 to move to the standard position that corresponds to the cleansing by the bidet function.

The wide cleansing key 212 is an operation switch which operates only after the bidet key 211 is pressed. With a press of the wide cleansing key 212, the controlling unit 114 has a spray opening of the nozzle 115 switched to a shower or enlarged opening so as to have warm water sprayed on a wider area than an area sprayed by the usual bidet function. This wide cleansing function is provided in response to the needs of women who wish to cleanse the menstrual blood off the wider area of the skin. When the stop key 213 is pressed during the cleansing by the bidet function, the controlling unit 114 stops the water supply to the cleansing nozzle 115 and moves the nozzle 115 to a predetermined position. In this way, the cleansing performance is terminated.

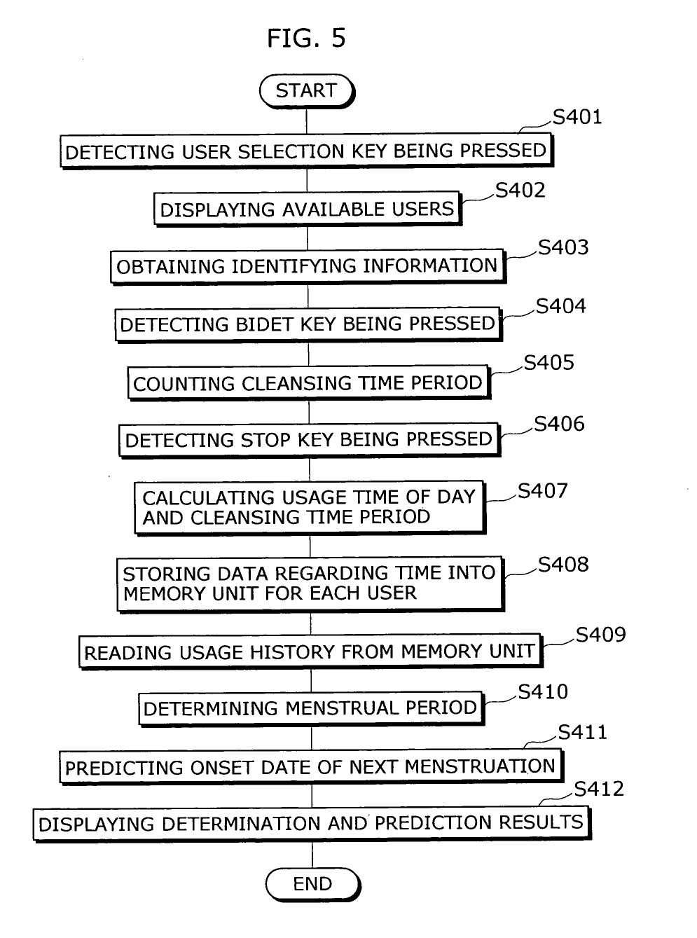

Next, an explanation is given for an operation that is performed by the menstrual cycle monitoring apparatus 100 shown in FIG. 1 while the user is using the bidet function, with reference to FIG. 5. FIG. 5 is a flowchart showing the operation performed by the menstrual cycle monitoring apparatus 100, from determining a menstrual cycle of the user to indicating the determination result to the user.

Before activating the bidet function, the user first presses the user selection key 201 in order to input the identifying information of the user. The user identifying unit 102 of the information processing unit 101 detects an operation of the user selection key 201 from the inputting unit 107 (S401). Then, the user identifying unit 102 directs the liquid crystal display 208 to display the user selection screen as shown in FIG. 3 to receive the input of the identifying information from the user (S402). Following this, the user identifying unit 102 obtains the identifying information to identify the user from the position of the cursor (i.e., the position of “∘”, for example) that is determined through the input by the user using the arrow and enter keys of the selection enter key 202 (S403). The user identifying unit 102 reads out the individual setting specified by the obtained identifying information from the memory unit 104, and notifies the controlling unit 114 of the read individual setting. The controlling unit 114, in turn, directs the cleansing nozzle 115 to move to a cleansing position that is stored in the notified individual setting.

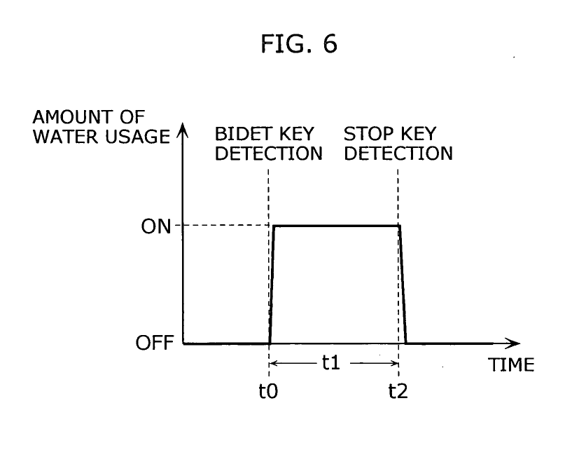

FIG. 6 is a graph representing a calculation method used by the menstrual cycle determining unit 115 for calculating a cleansing time period t0 spent by the user on cleansing using the bidet function. In the graph shown in FIG. 6, the horizontal axis represents time, while the vertical axis represents ON/OFF of the bidet function in relation to the amount of warm water or the like used during the cleansing by the bidet function. Here, as shown in FIG. 6, the amount of warm water sprayed during a cleansing by the general bidet function is constant per unit of time. The menstrual cycle determining unit 105 calculates the cleansing time period t0 taken per cleansing and stores the t0 into the memory unit 104 on a daily basis. Note that the cleansing time period t0 is a time length measured from when the bidet key 211 is pressed to when the stop key 213 is pressed. Upon receiving a notification from the controlling unit 114 that the user has pressed the bidet key 211 (S404), the menstrual cycle determining unit 105 obtains the current date as well as the current time of day indicated as a cleansing start time “t0” in FIG. 6 from the clock unit 103, and starts counting the cleansing time period t0 (S405). Then, upon receiving a notification from the controlling unit 114 that the user pressed the stop key 213 (S406), the menstrual cycle determining unit 105 obtains the current time indicated as a cleansing end time “t2” in FIG. 6 from the clock unit 103. The menstrual cycle determining unit 105 obtains the cleansing time period t0 by calculating a difference between the obtained current time, namely the cleansing end time t2, and the time obtained in step S404, namely the cleansing start time t0 (S407). After this, the menstrual cycle determining unit 105 accumulates, or additionally stores, the usage history including the calculated cleansing time period t0 as well as a usage time of day counted from t0 to t2 for each user in association with the identifying information of the user (S408).

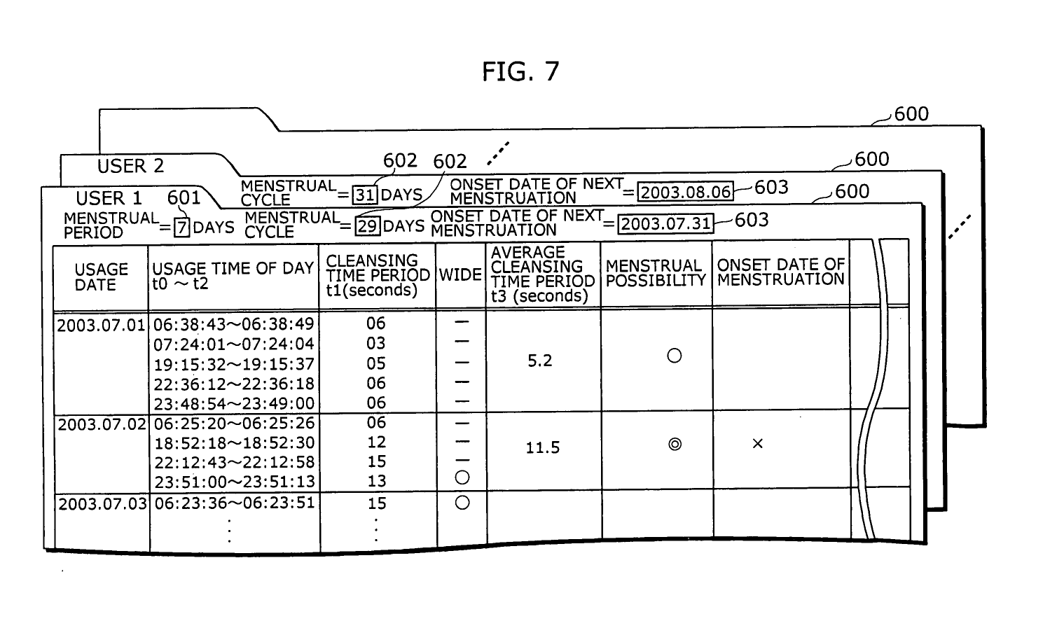

FIG. 7 shows an example of usage data 600 stored in the memory unit 104 shown in FIG. 1. As shown in FIG. 7, the usage data 600 is stored in the memory unit 104 for each user and it accumulates the above-mentioned usage history, including: usage date in year, month and day format; usage time of day counted between t0 and t2; cleansing time period t0; and information as to whether the wide cleansing function has been used. In addition to these sets of information, the usage data 600 also includes calculation results and determination results given by the menstrual cycle determining unit 105 and the menstrual cycle predicting unit 106. More specifically, these results are described in the corresponding columns of: average cleansing time period t3; menstrual possibility; onset date of menstruation; a length of menstrual period; a length of menstrual cycle; and onset date of next menstruation. A date in which the bidet apparatus 116 is used is described in the column of the usage date in year, month, and day format, while a time period during which the bidet function is used is described in the column of the usage time of day. The column of the cleansing time period t0 describes a time period between when the bidet key 211 is pressed and when the stop key 213 is pressed. The column of “wide” describes whether or not the wide cleansing key 212 is pressed during the cleansing by the bidet apparatus 116. Note here that when the wide cleansing key 212 is not pressed, “-” is written, while when it is pressed, “∘” is written. The column of the average cleansing time period t3 describes a value of an average time of cleansing time periods spent using the bidet apparatus 116, the average being calculated for each day. When the current date is written in the usage date of the usage data 600, the column of the average cleansing time period t3 describes an average value that is calculated from all the time periods spent so far on the current day using the bidet apparatus 116. That is, the calculation is made on the basis of a total usage time period including the first usage to the latest on that day.

The column of the menstrual possibility shows a level of the menstrual possibility of the current day. The level is indicated as “x”, “∘”, or “⊚”. The level shown in the column reflects a determination result given by the menstrual cycle determining unit 105 which determines the level of the menstrual possibility of the current day from the average cleansing time period t3 by reference to the table in the memory unit 104 that shows the determination standards. In the column of the onset date of menstruation, the onset is indicated as “x” which is a determination result given by the menstrual cycle determining unit 105. In a box of a menstrual period 601 provided on the margin of the usage data 600, a number of menstrual days is described as a determination result given by the menstrual cycle determining unit 105. In a box of a menstrual cycle 602, the length of the user's menstrual cycle is described by the number of days, which is calculated by the menstrual cycle predicting unit 106. In a box of an onset date of next menstruation, the date of the onset of the user's next menstruation is described as a prediction result given by the menstrual cycle predicting unit 106.

Getting back to the flowchart in FIG. 5, the menstrual cycle determining unit 105 reads out a bidet usage history of the user that is accumulated as shown in FIG. 7 from the memory unit 104 (S409). By reference to the read usage history, the menstrual cycle determining unit 105 determines the lengths of the user's past menstrual period and cycle (S410). On the basis of this determination result given by the menstrual cycle determining unit 105, the menstrual cycle predicting unit 106 predicts the date of the onset of the user's next menstruation (S411) and directs the liquid crystal display 208 to display the length of the user's menstrual cycle and the date of the onset of her next menstruation for a predetermined period of time (S412). Here, the processing is terminated.

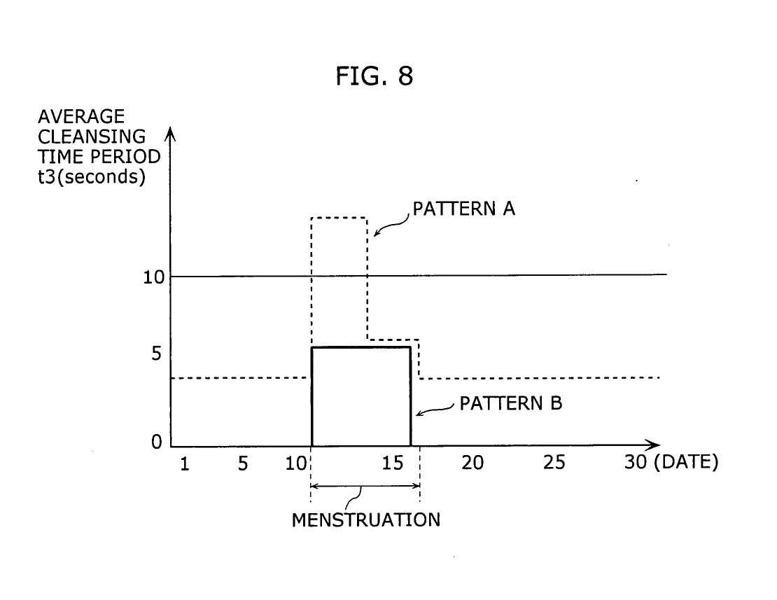

The following is a detailed explanation of the menstrual cycle determination processing performed by the menstrual cycle determining unit 105 in step S410 of the flowchart shown in FIG. 5, with reference to FIGS. 8 to 11.

FIG. 8 is a graph showing respective typical bidet usage patterns during menstruation and other days. In this graph, the vertical axis represents the average cleansing time period t3 while the horizontal axis represents the date. A graph in a dashed line shows fluctuations of the average cleansing time period t3 of a pattern A. A graph in a solid line shows fluctuations of the average cleansing time period t3 of a pattern B. It should be noted here that the menstrual period lasts from the 10^{th} to 17^{th} in this example. The length of time spent for the cleansing using the bidet function during menstruation is longer, as compared with the other days. This is because many women wish to cleanse off the menstrual blood deposited on the external vaginal region to preserve cleanliness and have a refreshed feeling. This usage pattern is shown as the pattern A in FIG. 8. The pattern A also indicates that the bidet function tends to be used even longer during two to three days following the onset of menstruation due to the relatively large amount of menstrual blood discharged during these days. On the other hand, there are also many women who use the bidet function only during the menstrual period, but not during the other days. This usage pattern is shown as the pattern B in FIG. 8. Keeping the attention on these different patterns A and B, the menstrual cycle determining unit 105 determines the user's menstrual period as well as her menstrual cycle on the basis of the variations in usage time period from day to day. Note that a normal menstrual period lasts five to seven days, and a normal menstrual cycle lasts 24 to 38 days. In accordance with these averages, the menstrual cycle determining unit 105 determines the menstrual period and cycle of the user on the basis of the user's average cleansing time period t3 over the last 35 days.

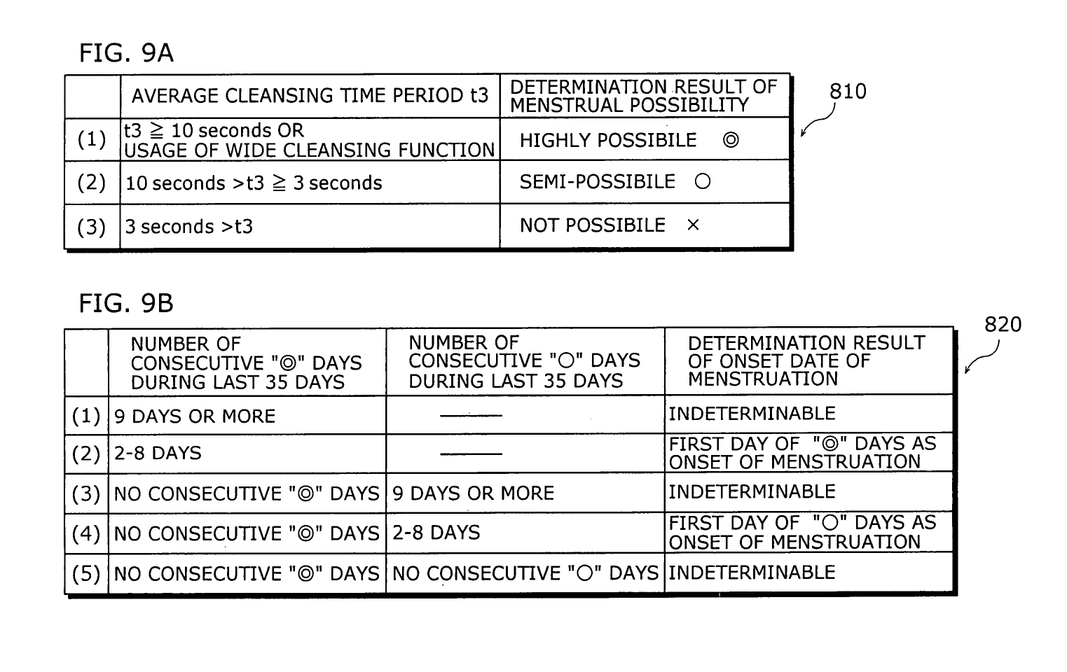

Next, with reference to FIG. 9, an explanation is given for a method used by the menstrual cycle determining unit 105 to determine the user's menstrual cycle of a predetermined time period. FIGS. 9A and 9B are tables showing determination standards that the menstrual cycle determining unit 105 refers to for determining the menstrual possibility for each day as well as the onset date of the past menstruation of the user. FIG. 9A is an example of a table 810 showing the determination standards that the menstrual cycle determining unit 105 refers to when determining the menstrual possibility for each day on the basis of the average cleansing time period t3. A detailed explanation of the table 810 in FIG. 9A is given as follows.

As indicated in the column (1), when the average cleansing time period t3 is 10 seconds or longer (i.e., during the menstrual period in the case of the pattern A shown in FIG. 8) or the wide cleansing function has been used, the menstrual possibility of the current day is judged to be “highly possible” and the determination result is indicated as “⊚”.

The column (2) describes a case where the average cleansing time period t3 is 3 seconds or longer but shorter than 10 seconds. Here, it is a menstrual day in the case of the pattern A, and it is not necessarily so in the case of the pattern B. Thus, the menstrual possibility of the current day is judged to be “semi-possible” and the determination result is indicated as “∘”.

As indicated in the column (3), when the average cleansing time period t3 is shorter than 3 seconds or the bidet function has not been used, the menstrual possibility of the current day is judged to be “not possible” and the determination result is indicated as “x”.

According to these determination standards shown in the table 810, the menstrual cycle determining unit 105 determines the menstrual possibility of the current day and describes the determination result into the corresponding column. To be more specific, when the average cleansing time period t3 described in the usage data 600 shown in FIG. 7 is 10 seconds or longer or the wide cleansing function has been used, the menstrual cycle determining unit 105 determines the menstrual possibility of the current day to be “highly possible” and describes “⊚” as the determination result into the column of the menstrual possibility in the usage data 600 of the user. When the average cleansing time period t3 is 3 seconds or longer but shorter than 10 seconds, the unit 105 determines the menstrual possibility of the current day to be “semi-possible” and describes “∘” as the determination result. When the average cleansing time period t3 is shorter than 3 seconds or the bidet function has not been used, the unit 105 determines the menstrual possibility of the current day to be “not possible” and describes “x” as the determination result.

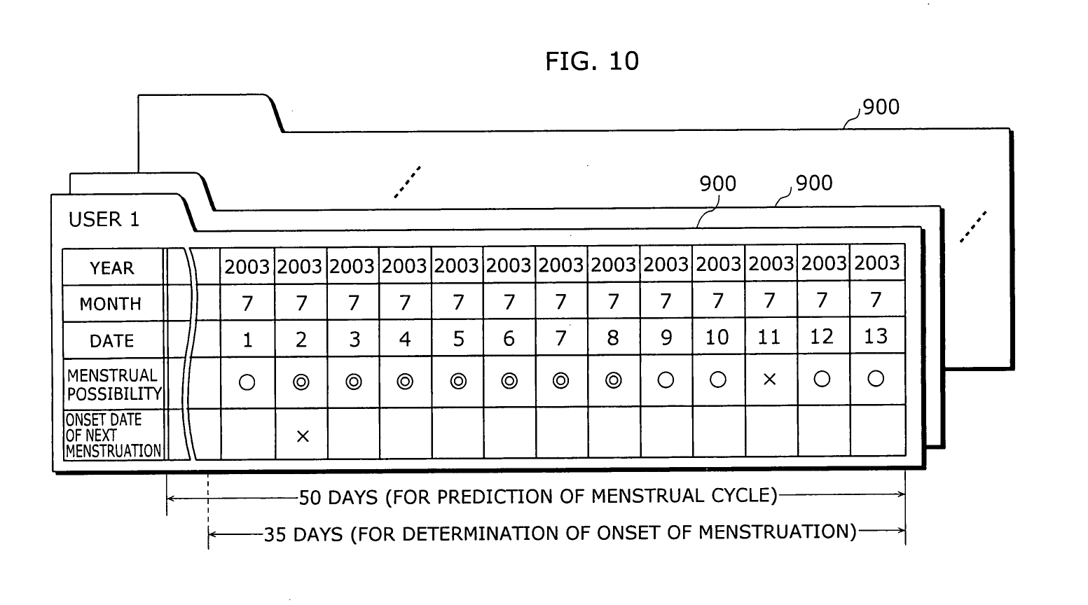

Next, the menstrual cycle determining unit 105 creates a menstrual calendar of the user in the memory unit 104 shown in FIG. 1 by reference to the column of the menstrual possibility included in the usage data 600, the menstrual calendar showing the menstrual possibility as “⊚”, “∘”, or “x” for each day. FIG. 10 is an example of a menstrual calendar 900 created in the memory unit 104. As shown in FIG. 10, the menstrual calendar 900 extracts the determination results described in the column of the menstrual possibility in the usage data 600 of FIG. 7 at least over the last 50 days from the current date, and the results are arranged in order by date. In addition, the menstrual calendar 900 includes a column into which the onset date of menstruation determined by the menstrual cycle determining unit 105 is indicated. By reference to the column of the menstrual possibility shown on the menstrual calendar 900 over the last 35 days from the current date, the menstrual cycle determining unit 105 reviews the consecutiveness of each mark (namely, “⊚”, “∘”, or “x”) showing the level of the menstrual possibility and determines the date of the onset of the user's menstruation, etc. in accordance with the consecutiveness. As the determination result, the menstrual cycle determining unit 105 describes “x” into the corresponding column of the onset date of the user's menstruation on the menstrual calendar 900.

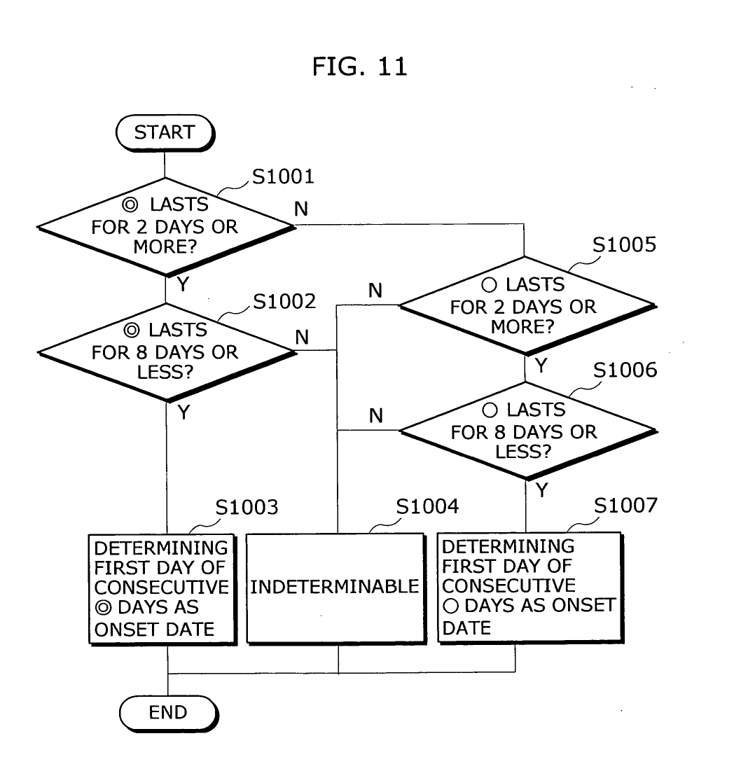

FIG. 9B is an example of a table 820 showing the determination standards that the menstrual cycle determining unit 105 refers to when determining the date of the onset of the user's menstruation on the basis of the menstrual calendar 900 shown in FIG. 10. FIG. 11 is a flowchart of an operation performed by the menstrual cycle determining unit 105 to determine the date of the onset of the user's menstruation by reference to the table 820 shown in FIG. 9B. A detailed explanation is given for the table 820 of FIG. 9B that shows the determination standards with respect to the menstrual possibility on the menstrual calendar 900 covering the last 35 days.

As indicated in the column (1), if “⊚” lasts for nine days or more during the last 35 days, the date of the onset of menstruation is judged to be “indeterminable”.

The column (2) describes a case where “⊚” lasts for two days or more but not more than eight days. In this case, the first day of these consecutive days is determined to be the date of the onset of the user's menstruation.

The column (3) describes a case where there is no consecutive “⊚”, and “∘” lasts for nine days or more. In this case, the date of the onset of menstruation is judged to be “indeterminable”.

As indicated in the column (4), if there is no consecutive “⊚”, and “∘” lasts for two days or more but not more than eight days, the first day of these consecutive days is determined to be the date of the onset of the user's menstruation.

Finally, the column (5) describes a case where there is no consecutive “⊚” nor “∘”. In this case, the date of the onset of menstruation is judged to be “indeterminable”.

According to these determination standards, the menstrual cycle determining unit 105 first judges whether “⊚” lasts for two days or more, by reference to the menstrual calendar 900 covering the last 35 days that is stored in the memory unit 104 (S1001). If so, the menstrual cycle determining unit 105 next judges whether “⊚” lasts for eight days or less (S1002). If so, the unit 105 determines the first day of these consecutive days to be the date of the onset of the user's menstruation, according to the standard indicated in the column (2) of the table 820 (S1003). In this way, the date of the onset of menstruation can be determined for the user who uses the bidet apparatus 116 in the manner of the pattern A. If judging that “⊚” lasts for nine days or more in step S1002, the unit 105 judges as “indeterminable” according to the standard indicated in the column (1) of the table 820 (S1004). If judging that “⊚” does not last in step S1001, the unit 105 next judges whether “∘” lasts for two days or more (S1005). If so, the unit 105 further judges whether “∘” lasts for eight days or less (S1006). If judging so, the unit 105 determines the first day of these consecutive days marked with “∘” to be the date of the onset of the user's menstruation, according to the standard indicated in the column (4) of the table 820 (S1007). In this way, the date of the onset of menstruation can be determined for the user who uses the bidet apparatus in the manner of the pattern B. Meanwhile, if judging that “∘” lasts for nine days or more in step S1006, the unit 105 judges as “indeterminable” according to the standard indicated in the column (3) of the table 820 (S1004). Also, if judging that “∘” does not last in step S1005, the unit 105 judges as “indeterminable” according to the standard indicated in the column (5) of the table 820 (S1004). Note that, however, the normal menstrual period lasting five to seven days cannot be determined simply on the basis of the number of consecutive days marked with “⊚” or “∘” in the following cases: where “∘” lasts less than two days and “⊚” lasts for two days or more but not more than four days; and where “⊚” lasts less than two days and “∘” lasts for two days or more but not more than four days. In such a case, on the assumption that the bidet function would be used only during two to three days after the onset of menstruation, i.e., during the amount of menstrual blood discharged is large, the menstrual cycle determining unit 105 determines the length of the menstrual period as five days uniformly, for example.

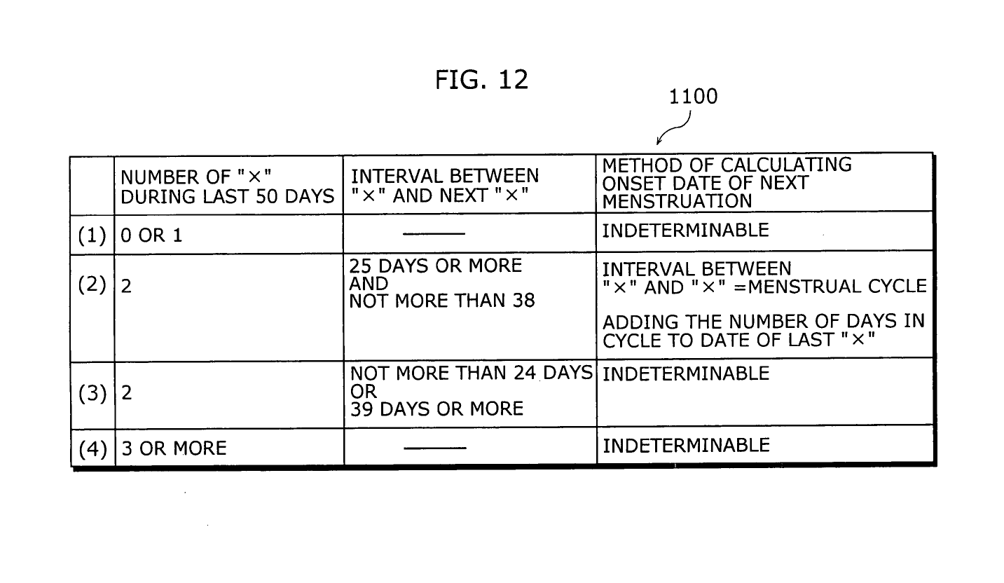

The following is a detailed explanation about processing performed by the menstrual cycle predicting unit 106 in steps S411 to S412 of the flowchart in FIG. 5 for predicting a next menstrual period of the user, with reference to FIGS. 12 to 14. The explanation is given for two cases where the menstrual cycle predicting unit 106 predicts the date of the onset of the user's next menstruation on the basis of: the determination result given by the menstrual cycle determining unit 105; and information regarding the date of the onset of the user's menstruation that is inputted by the user.

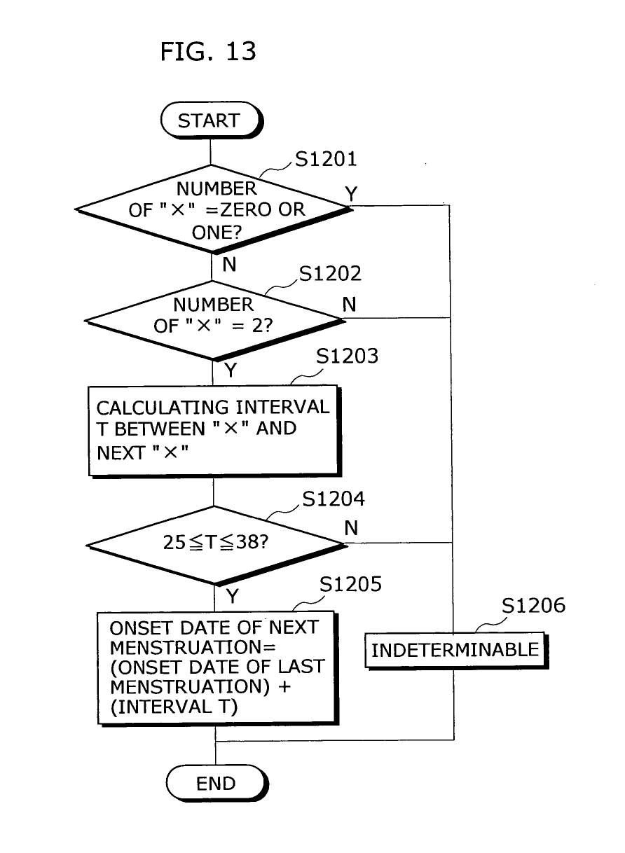

FIG. 12 is an example of a table 1100 showing the determination standards that the menstrual cycle predicting unit 106 refers to when predicting the date of the onset of the user's next menstruation on the basis of descriptions in the column of the onset date of menstruation on the menstrual calendar 900 covering the last 50 days. FIG. 13 is a flowchart of an operation performed by the menstrual cycle predicting unit 106 to predict the date of the onset of the user's next menstruation according to the table 1100 shown in FIG. 12. A detailed explanation is given for the descriptions in the table 1100 of FIG. 12 that are shown with respect to the onset date of menstruation on the menstrual calendar 900 covering the last 50 days.

As indicated in the column (1), if the number of “x” that indicates the onset date of menstruation is zero or one during the last 50 days, the onset date of the user's next menstruation is judged to be “indeterminable”.

The column (2) indicates a case where the number of “x” is two and an interval between a date of the onset of the firstly recorded menstruation and a date of the onset of the secondary recorded menstruation during the last 50 days is 25 days or more but not more than 38 days. In this case, the days that last between these two “x” marks is determined to be the user's menstrual cycle. The date obtained through adding the number of days in this menstrual cycle to the date of the onset of the secondary recorded (i.e., the latest) menstruation is predicted to be the onset date of the user's next menstruation.

The column (3) indicates a case where the number of “x” is two and an interval between a date of the onset of the firstly recorded menstruation and a date of the onset of the secondary recorded menstruation during the last 50 days is 24 days or less, or 39 days or more. In this case, the onset date of the user's next menstruation is judged to be “indeterminable”.

As indicated in the column (4), if the number of “x” is 3 or more, the onset date of the user's next menstruation is judged to be “indeterminable”.

According to these determination standards, the menstrual cycle predicting unit 106 first judges whether the number of “x” is zero or one, by reference to the column of the onset date of menstruation on the menstrual calendar 900 covering the last 50 days that is stored in the memory unit 104 (S1201). If not, that is, the number of “x” is two or more, the menstrual cycle predicting unit 106 next judges whether the number of “x” is two (S1202). If so, the unit 106 calculates the number of days lasting during an interval T (namely, the menstrual cycle) between these two “x” marks (S1203). Following this, the unit 106 judges whether the calculated value of the interval T satisfies an equation 25≦T≦38 (S1204). If judging so, the unit 106 calculates the date of the onset of the user's next menstruation by adding the obtained value of the interval T as the menstrual cycle to the latest date marked with “x” (S1205). Meanwhile, if the number of “x” is zero or one in step S1201, the unit 106 judges that the date of the onset of the user's next menstruation is “indeterminable” according to the standard indicated in the column (1) of the table 1100 (S1206). Also, if the number of “x” is not two in step S1202, meaning that it is three or more, the unit 106 judges that the date of the onset of the user's next menstruation is “indeterminable” according to the column (4) of the table 1100 (S1206). Moreover, if the value of the interval T does not satisfy the equation 25≦T≦38 in step S1204, meaning that the value satisfies an equation T≦24 or 39≦T, the unit 106 judges that the date of the onset of the user's next menstruation is “indeterminable” according to the column (3) of the table 1100 (S1206). This is to say, only if there is two “x” marks in the column of the onset date of menstruation during the last 50 days, the menstrual cycle predicting unit 106 calculates the interval T between these two “x” marks as the menstrual cycle. Moreover, only when the length of the calculated menstrual cycle is 24 to 38 days which is considered to be normal, the unit 106 calculates the date of the onset of the user's next menstruation by adding the number of days in the calculated menstrual cycle to the date of the onset of the latest menstruation. For the other cases, the unit 106 judges that the date of the onset of the user's next menstruation is “indeterminable”.

Accordingly, the explanation has been given for the case where the menstrual cycle predicting unit 106 predicts the user's menstrual cycle and the onset date of the user's next menstruation on the basis of the history of the onset date of the user's menstruation that was determined by the menstrual cycle determining unit 105. Instead of this processing, the user's menstrual cycle and the onset date of the user's next menstruation may be predicted on the basis of information inputted by the user indicating whether it is a menstrual day or not. For example, if the cleansing time period t0 using the bidet apparatus 116 was shorter than 10 seconds at the previous time and the cleansing time period t0 becomes 10 seconds or longer at the current time, the menstrual cycle determining unit 105 immediately directs the liquid crystal display 208 to display a question such as “Are you having your period today?” as well as display buttons of “Yes” and “No” in order for the user to input her answer to the question. If the user selects “Yes”, the menstrual cycle determining unit 105 determines the current day to be the date of the onset of the user's menstruation. Every time the user selects “Yes”, the menstrual cycle determining unit 105 records “x” indicating the onset of menstruation into the column of the onset date of menstruation on the menstrual calendar 900 stored in memory unit 104 and shown in FIG. 10. Meanwhile, the menstrual cycle predicting unit 106 reviews the column of the onset date of menstruation on the menstrual calendar 900 covering the last 50 days, and predicts the user's menstrual cycle and the onset date of the user's next menstruation following the steps of the flowchart in FIG. 13 by reference to the table 1100 shown in FIG. 12. In the case of such prediction on the basis of the information inputted by the user, the user has to take extra time and effort to input the answer as to whether she is currently having her period. However, the onset date of the user's next menstruation can be predicted with a higher degree of accuracy.

The menstrual cycle predicting unit 106 creates display data to show the determination result obtained through the stated processing to the user. This display data can be easily created by inserting a numeric obtained as a determination result into a character string that is previously stored corresponding to a detail of the determination in the memory unit 104. For example, for showing the date of the onset of the user's latest menstruation, the menstrual cycle predicting unit 106 directs the liquid crystal display 208 to display a message like “Your previous period started on 07/02.” Moreover, when the result is given as “indeterminable”, the menstrual cycle predicting unit 106 directs the liquid crystal display 208 to display a warning indicating the possibility of menstrual disorder as well as an advising message encouraging the user to use the bidet function. For instance, such message may be like, “Your menstrual cycle is indeterminable. Any menstrual problems? Using the bidet function during your period may enhance your personal hygiene and leave you feel refreshed.”

Accordingly, using the menstrual cycle monitoring apparatus 100 of the present embodiment, the menstrual cycle of an individual user can be determined only on the basis of her usage history for the bidet apparatus 116 mounted on the toilet fixture. Thus, no separate apparatuses need to be provided for the toilet fixture to examine excretion of bodily waste of the user. Also, the menstrual cycle monitoring apparatus 100 can provide the user with the determined menstrual period, menstrual cycle, and date of the onset of the user's next menstruation without upsizing the equipment to be mounted on the toilet fixture. Owing to this apparatus 100, the user can know about her menstrual cycle and the date of the onset of her next menstruation with no trouble, and can make use of such information for managing her physical condition during menstruation as well as for stocking up on menstrual sanitary products.

For a woman who is suffering from an unpleasant symptom caused by premenstrual syndrome (PMS) which remarkably emerges during the follicular phase, her uneasiness and impatience can be lessened by easily knowing from the information regarding the date of the onset of her next menstruation that she is in the phase of feeling this way. Moreover, she can ease the symptom by trying something for a refreshing change of pace or trying to lead a slow life without frustrations.

If the user knows how to predict her ovulation date from the date of the onset of her next menstruation according to a back calculation, her ovulation date can be easily calculated from the date of the onset displayed by the menstrual cycle monitoring apparatus 100. Knowing the ovulation date can be useful for a woman who practices birth control such as contraception or fertilization.

It should be noted here that an ovulation date predicting unit for predicting the user's ovulation date may be provided in the information processing unit 101 of the menstrual cycle monitoring apparatus 100 in addition to the construction shown in FIG. 1. The period lasting from ovulation to the onset of the next menstruation does not vary so much among women, and its length is constantly about 14 days or 14±2 days. On the basis of this, the ovulation date predicting unit predicts the user's ovulation date by calculating a date which comes 14 days before the onset date of the user's next menstruation that is predicted by the menstrual cycle predicting unit 106, and also predicts her fertile phase by calculating a period lasting between 16 and 12 days before the onset date of her next menstruation. The ovulation date predicting unit then directs the displaying unit 108 to display these prediction results.

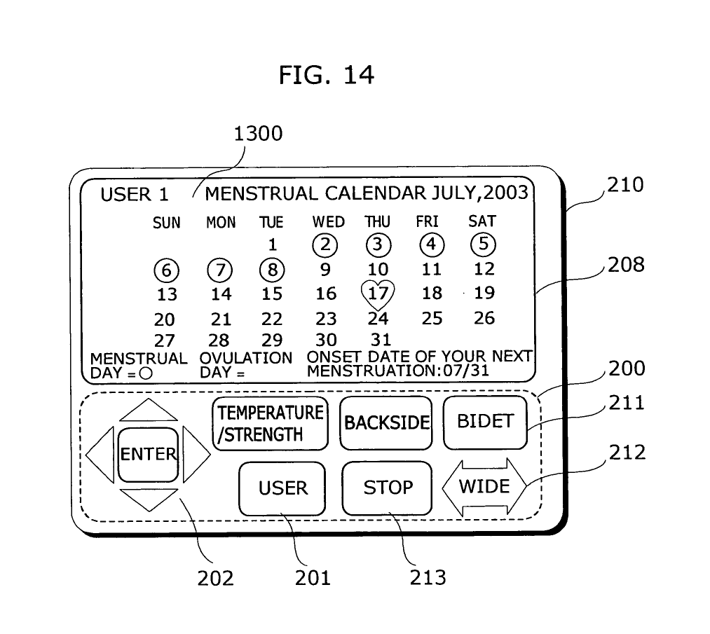

FIG. 14 is an example of a calendar 1300 showing the prediction results given by the ovulation date predicting unit which is additionally provided for the menstrual cycle monitoring apparatus 100 shown in FIG. 1. As shown in FIG. 14, the calendar 1300 of “JULY, 2003” of the “USER 1” is displayed on the liquid crystal display 208. On the calendar 1300, for example, the dates from the 2^{nd} to 8^{th} are respectively encircled by a “∘” mark which indicates a menstrual day. This determination result is given by the menstrual cycle determining unit 105. Moreover, the 17^{th} is encircled by a heart-shaped mark which indicates an ovulation day, as the prediction result given by the ovulation date predicting unit. On the lower right part of the liquid crystal display 208, the determination result by the menstrual cycle predicting unit 106 is displayed as “The onset date of your next menstruation is 07/31.”

According to this construction, the menstrual cycle monitoring apparatus 100 predicts not only the date of the onset of the user's next menstruation but the user's ovulation date as well using the ovulation date predicting unit. Therefore, the user can easily know her ovulation date without having to count backward on the calendar. On account of such information regarding the ovulation date, a woman who does not wish to become pregnant will pay more attention to birth control during her ovulation phase at which she is most fertile. On the other hand, a woman who wishes to become pregnant can increase her chance of pregnancy by having intercourse during her ovulation phase. In addition, the user can use the predicted ovulation date as a guide to remember a starting date of urine test that is performed using an ovulation test agent. By doing so, the user can avoid a situation in which she forgets about the urine test and lets the test agent go to waste.

Moreover, the menstrual cycle predicting unit 106 may direct the liquid crystal display 208 to display a mark indicating a length of the cleansing time period for each date on the calendar of the current month. The lengths may be indicated as “⊚”, “∘”, and “x”, meaning from longer to shorter. By reference to the mark assigned for each date, the user may determine the date of the onset of her menstruation by looking back on her bidet usage history for herself.

In the stated embodiment, the determination and prediction results given by the menstrual cycle determining unit 105, the menstrual cycle predicting unit 106, and the ovulation date predicting unit are displayed on the liquid crystal display 208. However, a speaker may be provided so that the user can be informed of these results through audio assist.

As described in the stated embodiment, the remote controller 210 shown in FIG. 3 is provided integrally with the operating unit 110 of the bidet apparatus 116 and the inputting unit 107 and the displaying unit 108 of the menstrual cycle monitoring apparatus 100. Also, the main unit 220 of the bidet apparatus 116 is provided integrally with the controlling unit 114 (as well as the operating unit 110) of the bidet apparatus 116 and the information processing unit 101 of the menstrual cycle monitoring apparatus 100. However, the menstrual cycle monitoring apparatus of the present invention is not limited to this construction. For example, the menstrual cycle monitoring apparatus 100 may be provided in the remote controller 210. In this case, the information processing unit 101 performs the stated determination processing by directly detecting a signal outputted from an operation switch of the operating unit 110 instead of receiving a notification from the controlling unit 114 that the operation switch has been pressed.

Moreover, the bidet apparatus 116 may be provided with a communication function for notifying the menstrual cycle monitoring apparatus 100 of an operation performed on the operating unit 110 by the user, via a home network achieved by power lines or the like or via a communication network such as a cellular phone or public telephone network. In this case, the menstrual cycle monitoring apparatus 100 may be provided for a personal computer (PC), server apparatus, cellular phone, or personal digital assistant (PDA) that is set outside the toilet fixture and located inside or outside the housing. Here, the inputting unit 107 and the displaying unit 108 of the menstrual cycle monitoring apparatus 100 can be realized respectively by an existing keyboard and a liquid crystal display which may be provided for the PC, server apparatus, cellular phone, or PDA. The information processing unit 101 can be realized by a program that is previously installed in a memory unit of the PC, server apparatus, cellular phone, or PDA and is executed by a corresponding CPU.

In the stated embodiment, the nozzle position setting screen 300 shown in FIG. 4 is displayed, for example, after the user selection key 201 is pressed for two seconds or longer following the input of the identifying information from the user selection screen shown in FIG. 3. However, the present invention is not limited to this. The nozzle position setting screen 300 may be displayed after the user selection key 201 is pressed long regardless of before or after the identifying information is inputted. Here, the period of time during which the key 201 is pressed long is not limited. In the case where the nozzle position setting screen 300 is displayed before the identifying information is inputted, the user selection screen may be displayed after the nozzle position is selected on the screen 300 and the selection enter key is pressed, so that the user can then input the identifying information. Note that the screen 300 may be displayed after the user pressed the user selection key 201 a predetermined number of times, instead of pressing it for two seconds or longer.

As described in the stated embodiment, the menstrual cycle determining unit 105 calculates the cleansing time period t0 taken from when the user presses the bidet switch 111 to when the user presses the stop switch 113 and accumulates the cleansing time period t0 into the memory unit 104. On the basis of the accumulated time period t0, the menstrual cycle determining unit 105 determines the date of the onset of the user's menstruation, the user's menstrual cycle, and the date of the onset of the user's next menstruation. However, the present invention is not limited to this. For example, the menstrual cycle determining unit 105 may detect the amount of water sprayed from the cleansing nozzle 115 between when the bidet switch 111 is pressed and when the stop switch 113 is pressed, and may determine the date of the onset of the user's menstruation, etc. according to the user's water usage history.

In the stated embodiment, the menstrual cycle determining unit 105 determines the menstrual possibility for each day on the basis of the average cleansing time period t3 of the current day. However, the present invention is not limited to this, and the unit 105 may determines the menstrual possibility on the basis of the maximum length of the cleansing time period t0 of the current day, for example. To be more specific, if the maximum length of the cleansing time period t0 of the current day is 10 seconds or longer, the menstrual possibility may be judged to be “highly possible”. If the maximum length of the cleansing time period t0 of the current day is 3 seconds or longer but shorter than 10 seconds, the menstrual possibility may be judged to be “semi-possible”. If the maximum length of the cleansing time period t0 of the current day is shorter than 3 seconds, the menstrual possibility may be judged to be “not possible”. Here, if the cleansing time period t0 becomes 10 seconds or longer at least once during the current day, the menstrual possibility may be judged to be “highly possible” in accordance with the length at that one time.

Moreover, in consideration of the pattern B shown in FIG. 8, if the average cleansing time period t3 is shorter than 3 seconds but the cleansing time period t0 is more than 0, the menstrual cycle determining unit 105 may judge that the menstrual possibility of the current day is “highly possible”. Here, for the case of the user who uses the bidet apparatus 116 in the manner of the pattern A, such judgment as “highly possible” will last for nine days or more and the date of the onset of menstruation is judged to be “indeterminable”. For this reason, when this happens, the method of determining the menstrual possibility may be switched to the determination method using the table 810 shown in FIG. 9A from then on.

Moreover, as another preferred embodiment where the user uses the bidet apparatus in the manner of the pattern B, a menstrual day may be determined according to whether or not the bidet function has been used, without calculation of the average cleansing time period. In this case, when the bidet switch 111 is pressed, information that the bidet function has been used is accumulated into the memory unit 104 together with information such as the date and time when the bidet function was used. Here, on the basis of the information accumulated in the memory unit 104, the menstrual cycle determining unit 105 determines the menstrual possibility of the current day as “highly possible”, on which the bidet function is judged to have been used.

In the stated embodiment, the menstrual possibility for each day is indicated by the mark “⊚”, “∘”, or “x”. However, these marks do not necessarily have to be used, and any expressions can be used as long as they can show the levels of the possibility. For example, “highly possible”, “semi-possible” and “not possible” may be indicated respectively as three numbers “3”, “2”, and “1”. Alternatively, each level may be indicated as a different alphabetical letter, or a combination of a number and an alphabetical letter. Moreover, although the menstrual possibility has been indicated in the three levels, the number of levels is not limited.

As described in the stated embodiment, if the wide cleansing function is used, the menstrual possibility of the current day is judged to be “highly possible”. However, the menstrual possibility may be judged only according to the time period taken for the cleansing using the bidet function regardless of whether the wide cleansing function has been used or not.

In the stated embodiment, when “⊚” or “∘” lasts for two days or more but not more than four days, the menstrual cycle determining unit 105 determines the length of the menstrual period as five days uniformly. The present invention is not limited to this, and the menstrual period may be any length of days as long as it is five to seven days. In addition, when “⊚” lasts for two days or more but not more than four days, the menstrual cycle determining unit 105 may add the number of following consecutive “∘” to the number of “⊚” and determine the number of these days within five to seven days to be the menstrual period.

Moreover, in the stated embodiment, when “⊚” does not last two days or more, the menstrual cycle determining unit 105 determines the date of the onset of the user's menstruation according to the number of consecutive “∘”. However, the date of the onset of the user's menstruation may be determined on the basis of the number of consecutive days which are respectively indicated as “⊚” or “∘”. Accordingly, although the determination result for “x-x-∘-⊚-∘-⊚-∘-x-x” would be “indeterminable” in the stated embodiment, the menstrual cycle determining unit 105 can correctly determine the date of the onset of the user's menstruation. Similarly, for the case of “x-x-⊚-∘-∘-∘-x-x”, although the first date of the consecutive “∘” would be determined incorrectly to be the date of the onset of the user's menstruation in the stated embodiment, the menstrual cycle determining unit 105 will not give such incorrect result.

For determining the date of the onset of the user's menstruation in the stated embodiment, the menstrual cycle determining unit 105 refers to the history of the menstrual possibility covering the last 35 days. Meanwhile, for predicting the date of the onset of the user's next menstruation in the stated embodiment, the menstrual cycle predicting unit 106 refers to the history of the onset date of menstruation covering the last 50 days. However, the number of days needs not to be 35 or 50, and the number of days to be subject to reference may be any number as long as it is 35 days or more. For example, the history covering 40 days may be referred to for both such determination and prediction.

When the interval T from the onset date of the firstly recorded menstruation to the onset date of the secondly recorded menstruation does not satisfy the equation 25≦T≦38, the judgment result is given as “indeterminable” in the stated embodiment. However, the onset date of the next menstruation may be determined in consideration of a possible determination error made by the menstrual cycle determining unit 105. For example, suppose that the possible determination error made by the unit 105 as to the date of the onset of menstruation is zero to three days. When the interval T satisfies an equation 22≦T≦24, the menstrual cycle may be determined as 25 days and, on the basis of this, the date of the onset of the next menstruation may be calculated. Also, when the interval T satisfies an equation 39≦T≦41, the menstrual cycle may be determined to be 38 days and, on the basis of this, the date of the onset of the next menstruation may be calculated.

In the stated embodiment, the menstrual cycle determining unit 105 judges the menstrual possibility of the current day to be: “not possible” when the average cleansing time period t3 is shorter than 3 seconds; “semi-possible” when the t3 is 3 seconds or longer but shorter than 10 seconds; and “highly possible” when the t3 is 10 seconds or longer. However, the thresholds used for such determination is not limited to 3 seconds and 10 seconds. These thresholds may be set as needed in accordance with the amount of warm water sprayed per unit time from the cleansing nozzle 115 of the bidet apparatus 116 or statistical data regarding the amount of water used per one time for a cleansing by the bidet function. Moreover, the user may previously set her optimum thresholds manually.

In the stated embodiment, every time the user uses the bidet apparatus 116, the menstrual cycle monitoring apparatus 100 accumulates the user's usage history so that the menstrual possibility, the date of the onset of the user's menstruation, and the date of the onset of the user's next menstruation are all determined on the basis of the accumulated usage history of the user. However, these determinations may be performed only at the user's first time usage of the bidet apparatus 116 each day, on the basis of the user's usage history covering the past days until the previous day. Besides this first usage record of each day, the usage data 900 may be updated only when a new record regarding the bidet usage is accumulated into the memory unit 104. Owing to this, as compared to the case where the above determination and prediction are performed for each time, electric power consumption by the bidet apparatus 116 and the menstrual cycle monitoring apparatus 100 can be reduced.

In the stated embodiment, each user previously sets a number as identifying information to uniquely identify the user, so that the user can input her identifying information by selecting her pre-set number. However, the user identification method of the present invention is not limited to this. For example, a scale may be built in the toilet seat, and each user may previously store her weight into the ovulation cycle monitor system 100. Every time the user's weight is measured by the built-in scale when she sits on the toilet seat, the information processing unit 113 compares this measured weight with the weight that is pre-stored for the user. As the comparison result, if a difference between the measured weight and the pre-stored weight falls within a predetermined range (±0.5 kg, for instance), the measured weight is judged to agree with the pre-stored weight. Accordingly, the user can be identified on the basis of the pre-stored weight. In the case where two or more users have the similar weight, with a difference being about 1 kg, it is preferable to use this method in combination with the stated method that uses a pre-stored number assigned for each user.

Moreover, besides the weight, the user may be identified by her height, for example. In this case, a sensor for measuring the user's height is set at the bathroom door, and the measured height may be compared with the height previously stored for the user. As is the case of the above weight method, if two or more users have the similar height, it is preferable to use this height method in combination with the method that uses a pre-stored number assigned for each user.

Furthermore, besides the methods based on the weight and height, the weight distribution over the toilet seat may be stored for each user. Every time a user sits on the toilet seat, the pressure distribution over the toilet seat may be measured for the user identification.

Alternatively, the user may be identified by her fingerprints or profile of the face. Moreover, a circuit may be provided for an accessory that a user wears, such as a wristwatch and a ring, or for a device that the user always carries, such as a cellular phone. With this circuit, the user's identifying information may be transmitted to the ovulation cycle monitor system 100 via wireless or cellular phone communication. Also, identifying information that is readable by radio waves may be written onto an electronic tag, such as an RFID tag or a wireless IC tag, and the ovulation cycle monitor system 100 may read the identifying information by emitting radio waves.

The menstrual cycle monitoring apparatus of the present invention is useful as a toilet apparatus such as a toilet seat or toilet bowl which is integrally provided with a bidet apparatus. Moreover, the menstrual cycle monitoring apparatus of the present invention is useful as a remote controller of a bidet apparatus, a personal computer, a server apparatus, a personal digital assistant (PDA), or a cellular phone that has a communication function.

Although the present invention has been fully described by way of examples with reference to the accompanying drawings, it is to be noted that various changes and modifications will be apparent to those skilled in the art. Therefore, unless otherwise such changes and modifications depart from the scope of the present invention, they should be constructed as being included therein.

1. A menstrual cycle monitoring apparatus that determines a menstrual cycle of a user, comprising:
a usage quantity measuring unit operable to measure a usage quantity of a bidet apparatus that cleanses a vaginal area of the user;
a history accumulating unit operable to accumulate a history of the measured usage quantity for each date; and
a menstrual cycle determining unit operable to determine the menstrual cycle of the user on the basis of the history of the measured usage quantity accumulated in said history accumulating unit.

2. The menstrual cycle monitoring apparatus according to claim 1,
wherein the usage quantity is an amount of water used by said bidet apparatus for a cleansing,
said usage quantity measuring unit includes a water meter operable to measure the amount of water used for the cleansing,
said history accumulating unit accumulates a history of the measured amount of water,
said menstrual cycle determining unit includes a menstrual day determining unit operable to determine a possible menstrual day by reference to the history of the measured amount of water, and
said menstrual cycle determining unit determines the menstrual cycle of the user on the basis of a result given by said menstrual day determining unit.

3. The menstrual cycle monitoring apparatus according to claim 1,
wherein the usage quantity is a cleansing time period taken by said bidet apparatus for a cleansing,
said usage quantity measuring unit includes a timer operable to count the cleansing time period,
said history accumulating unit accumulates a history of the counted cleansing time period,
said menstrual cycle determining unit includes a menstrual day determining unit operable to determine a possible menstrual day by reference to the history of the counted time period, and
said menstrual cycle determining unit determines the menstrual cycle of the user on the basis of a result given by said menstrual day determining unit.

4. The menstrual cycle monitoring apparatus according to claim 3,
wherein said menstrual day determining unit determines a day, as a possible menstrual day, on which the cleansing time period exceeding a predetermined threshold has been accumulated in said history accumulating unit.

5. The menstrual cycle monitoring apparatus according to claim 3,
wherein said menstrual day determining unit includes a calculating unit operable to calculate an average of cleansing time periods accumulated for one day in said history accumulating unit, and
said menstrual day determining unit determines a day, as a possible menstrual day, whose calculated average exceeds a predetermined threshold.

6. The menstrual cycle monitoring apparatus according to claim 3,
wherein said menstrual cycle determining unit further includes an onset date determining unit operable to determine a date of an onset of a menstruation of the user by reference to the history in accordance with a number of consecutive days that have been determined as possible menstrual days by said menstrual day determining unit, and
said menstrual cycle determining unit determines the menstrual cycle of the user on the basis of a result given by said onset date determining unit.

7. The menstrual cycle monitoring apparatus according to claim 6,
wherein said menstrual cycle determining unit determines a first day of the consecutive possible menstrual days as the date of the onset of the menstruation of the user when the number of the consecutive possible menstrual days is two or more but not more than eight.

8. The menstrual cycle monitoring apparatus according to claim 6,
wherein said menstrual cycle determining unit determines the menstrual cycle of the user in accordance with a length of an interval between a date of an onset of a menstruation and a date of an onset of a next menstruation, with both of these dates having been accumulated in the history and having been determined by said onset date determining unit.

9. The menstrual cycle monitoring apparatus according to claim 8,
wherein said menstrual cycle determining unit determines the interval as the menstrual cycle of the user when the length of the interval is 25 days or more but not more than 38 days.

10. The menstrual cycle monitoring apparatus according to claim 1, further comprising
an onset date predicting unit operable to predict a date of an onset of a future menstruation of the user on the basis of a result given by said menstrual cycle determining unit.

11. The menstrual cycle monitoring apparatus according to claim 10,
wherein said onset date predicting unit predicts the date of the onset of the future menstruation of the user by adding a number of days in the determined menstrual cycle to a date of an onset of a latest menstruation accumulated in the history.

12. The menstrual cycle monitoring apparatus according to claim 10, further comprising
an ovulation date predicting unit operable to predict a future ovulation date of the user on the basis of a prediction result given by said onset date predicting unit.

13. The menstrual cycle monitoring apparatus according to claim 1,
wherein said menstrual cycle determining unit includes a menstrual day determining unit operable to determine a day, as a possible menstrual day, on which the usage quantity has been accumulated as a value other than “◯” in the history, and
said menstrual cycle determining unit determines the menstrual cycle of the user on the basis of a result given by said menstrual day determining unit.

14. The menstrual cycle monitoring apparatus according to claim 1, further comprising
a displaying unit operable to display a result given by said menstrual cycle determining unit to the user.

15. The menstrual cycle monitoring apparatus according to claim 1, further comprising
a receiving unit operable to receive an input of identifying information from the user to uniquely identify the user,
wherein said history accumulating unit accumulates the history of the measured usage quantity for each identified user, and
said menstrual cycle determining unit determines the menstrual cycle of the identified user on the basis of the history of the measured usage quantity accumulated in said history accumulating unit.

16. A menstrual cycle monitoring method that determines a menstrual cycle of a user, comprising:
a step of measuring a usage quantity of a bidet apparatus that cleanses a vaginal area of the user;
a step of accumulating a history of the measured usage quantity for each date; and
a step of determining the menstrual cycle of the user on the basis of the history of the measured usage quantity accumulated in said history accumulating step.

17. A program that is used for a menstrual cycle monitoring apparatus that determines a menstrual cycle of a user, causing a computer to function as:
a usage quantity measuring unit operable to measure a usage quantity of a bidet apparatus that cleanses a vaginal area of the user;
a history accumulating unit operable to accumulate a history of the measured usage quantity for each date; and
a menstrual cycle determining unit operable to determine the menstrual cycle of the user on the basis of the history of the measured usage quantity accumulated in said history accumulating unit.

18. A computer-readable recording medium that records a program used for a menstrual cycle monitoring apparatus that determines a menstrual cycle of a user, the program causing a computer to function as:
a usage quantity measuring unit operable to measure a usage quantity of a bidet apparatus that cleanses a vaginal area of the user;
a history accumulating unit operable to accumulate a history of the measured usage quantity for each date; and
a menstrual cycle determining unit operable to determine the menstrual cycle of the user on the basis of the history of the measured usage quantity accumulated in said history accumulating unit.

19. A toilet apparatus comprising one of a toilet seat and a toilet bowl on which a menstrual cycle monitoring apparatus operable to determine a menstrual cycle of a user is mounted, comprising:
a usage quantity measuring unit operable to measure a usage quantity of a bidet apparatus that cleanses a vaginal area of the user;
a history accumulating unit operable to accumulate a history of the measured usage quantity for each date; and
a menstrual cycle determining unit operable to determine the menstrual cycle of the user on the basis of the history of the measured usage quantity accumulated in said history accumulating unit.

20. A menstrual cycle monitoring apparatus that determines a menstrual cycle of a user, comprising:
a history accumulating unit operable to accumulate a usage history of a bidet apparatus that cleanses a vaginal area of the user; and
a menstrual cycle determining unit operable to determine the menstrual cycle of the user on the basis of the usage history accumulated in said history accumulating unit.
